(19) United States
(12) Patent Application Publication
(10) Pub. No.: US 2005/0228204 A1
(43) Pub. Date: Oct. 13, 2005

(54) Process for the preparation of xylene

(76) Inventor: Leo Ernest Manzer, (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 10/997,219

(22) Filed: Nov. 24, 2004

Related U.S. Application Data
(60) Provisional application No. 60/525,005, filed on Nov. 25, 2003.

(51) Int. Cl. C07C 2/64

(52) U.S. Cl. 585/446

(57) ABSTRACT

The present invention relates to a process for the preparation of xylene from diisobutylenes.

This application claims the benefit of U.S. Provisional Application No. 60/525,005, filed on Nov. 25, 2003, which is incorporated in its entirety as a part hereof for all purposes.

### FIELD OF THE INVENTION

The present invention relates to the production of xylene by the catalytic dehydrogenation of diisobutylene. The catalysts used in such process are, for example, those containing chromium or platinum.

### BACKGROUND

Para-xylene (p-xylene) is a useful aromatic material, especially for the production of terephthalic acid, which is used as a monomer in the production of polyester. It is therefore desirable to produce xylene, particularly para-xylene, in relatively high yields.

A primary commercial source of low molecular weight aromatics (i.e., benzene, toluene and xylene) is extraction from catalytic reformate, which is produced in petroleum refining for making high-octane gasoline. Reformate may contain from 20 to 30 percent of C₆ to C₈ aromatics. High purity aromatics can be removed only by selective extraction because of the overlapping boiling points of these aromatics with other hydrocarbons present in the reformate. Such processes are complex and costly, and isolation of the para-xylene isomer adds further complexity.

Separation of para-xylene from its isomers is usually done in one of two ways. The more recent method is to use an elaborate multi-valve absorption process using molecular sieves. An older method, still used, is multi-stage fractional crystallization at low temperatures to recover a pure para-xylene fraction. This includes the Badger/Niro and PAREX processes. Descriptions of these methods and others can be found in Report PERP 01/02-7, Xylenes, from Nexant Chem Systems, which is incorporated in its entirety as a part hereof for all purposes.

A major problem with separation schemes such as described above is that the para isomer of the three possible xylene isomers is present in only about 20% of the equilibrium mixture. Hence, large volumes of undesired materials are passed through either of the above separation processes to obtain the relatively minor amounts of para-xylene present. These processes suffer from the disadvantage of the need for costly and elaborate separation procedures.

Manufacture of aromatic hydrocarbons from acyclic alkanes or acyclic alkenes is known in the art. For example, U.S. Pat. No. 3,202,725 discloses a process for the manufacture of xylene containing greater than 95% of the commercially desirable para isomer. The process involves feeding to a catalytic dehydrogenation zone various hydrocarbon feeds that include isooctane, diisobutylene, and a mixture of isobutane and isobutylene. The dehydrogenation catalyst constitutes 15 to 25% chromium oxide (Cr₂O₃) on an alumina support composed essentially of eta-alumina. The yield of para-xylene per-pass in the aromatization step is low because of the ease with which the trimethylpentenes are cracked to isobutylene under the reaction conditions. A large recycle stream of the isobutylene is sent back to an acid dimerization step to produce additional trimethylpentane. Also, the disclosed process is performed at sub-atmospheric pressures, generally in the range of 5˜30 in Hg absolute (0.17˜1 atm).

U.S. Pat. No. 6,600,081 describes a process for the dehydrocyclization of trimethypentane to p-xylene using catalysts such as chromium-containing catalysts. This process not only involves various separations and isolations, it also starts with a material that is generally relatively expensive. It is, for example, generally difficult to obtain high yields of 2,2,4-trimethylpentane via a typical process such as the alkylation of isobutylene with isobutane.

A need thus remains for a process to make xylene that is efficient, avoids costly steps such as isolation and separation, and favors production of the para isomer. The present invention meets such need by providing a process to make xylene that uses as the starting material diisobutylene, which can be prepared easily and in high yield from variety of sources such as isobutylene.

### SUMMARY OF THE INVENTION

One embodiment of this invention is a non-oxidative process for the manufacture of xylene by (a) feeding to a reactor a reactor feed comprising diisobutylene, and a diluent gas selected from the group consisting of methane, ethane and mixtures thereof; and (b) contacting, in the vapor phase, the reactor feed with a dehydrogenation catalyst in a reactor to produce a stream of reactor effluent that comprises xylene.

A variety of dehydrogenation catalysts may be used such as those containing chromium and/or platinum. The xylene that is recovered from the reactor effluent may be purified by crystallization to increase the content of the para isomer, and, if desired, the para-xylene may be used to make terephthalic acid, which in turn can be used to make polyester.

The process provides for recycling unreacted diisobutylene, and for recovering from the effluent species such as isobutylene and isobutane that can be dimerized to make diisobutylene. If desired, isobutane can be fed from an external source to the same dehydrogenation reactor to prepare isobutylene.

### BRIEF DESCRIPTION OF THE DRAWING

FIG. 1 is a schematic flow diagram for one embodiment of this invention.

### DESCRIPTION OF VARIOUS EMBODIMENTS OF THE INVENTION

This invention provides a non-oxidative process for the manufacture of xylene from diisobutylene. A non-oxidative process is a process that is run in the substantial absence of, and preferably in the absence of, oxygen. Oxygen is substantially absent from the reaction system when hydrogen is generated on an essentially quantitative basis as a removable by-product of the reaction rather than forming water. A non-oxidative process is provided by reducing the content of oxygen in the feed stream to an insignificant level such as less than about 1.5 mole percent of the total feed stream, preferably less than about 0.5 mole percent thereof, more preferably only a trace amount as an impurity, and is most preferably provided by completely excluding oxygen from the feed stream to the reactor. A non-oxidative process is provided by not using a feed containing air or oxygen, and by preventing air intrusion into the process through the careful construction and maintenance of tight, well-sealed equipment.

The absence of oxygen from the feed stream is, of course, to be distinguished from the use of oxygen between production runs to regenerate a catalyst. When oxygen is used for such purpose, the reactor is purged with an inert gas such as nitrogen before the next production run.

Xylene, as referred to herein, includes all three of the ortho, meta and para isomers thereof and/or mixtures of any two isomers. Where a reference is to a particular isomer such as the para isomer, that will be indicated. Diisobutylene (DIB), as referred to herein, includes all forms of trimethylpentenes and dimethylhexenes.

The process of this invention, which is preferably done in a series of steps, takes advantage of the ready availability of DIB, which may for example be easily prepared from isobutylene in high yield. The DIB is readily converted to xylene in a dehydrocyclization step, which may be performed in a dehydrogenation reactor in the presence of an inert diluent.

A dehydrogenation catalyst is present in the dehydrogenation reactor used in this invention, and such catalyst may be prepared, for example, from chromium and/or platinum, preferably chromium. It is preferred that the catalyst be supported. The catalyst can be promoted or treated with metals selected from the group consisting of iron, tin, and tungsten. Preferably, the catalyst also contains at least one metal from Groups 1 and 2 (i.e., Na, K, Rb, Cs, Mg, Ca, Sr and Ba).

The catalyst is prepared by combining the component(s) with a refractory inorganic oxide support material, particular examples of which are alumina (especially eta-alumina) and zirconia. The metal(s) can be combined or intimately associated with a porous inorganic support or carrier by various known techniques such as ion-exchange, coprecipitation with the support (e.g., alumina) in the sol or gel form, and the like. For example, the catalyst can be formed by adding together suitable reagents such as salts of the required metal(s) and ammonium hydroxide or ammonium carbonate, and a salt of aluminum such as aluminum chloride or aluminum nitrate to form aluminum hydroxide. The aluminum hydroxide containing the salts can then be treated with the alkali or alkaline earth, heated, dried, formed into pellets or extruded, and then calcined.

Alternatively, the metal(s) can be deposited on a previously pilled, pelleted, beaded, extruded or sieved particulate support material by the impregnation technique. Porous refractory inorganic oxides in dry or solvated state are contacted, either alone or admixed, or otherwise incorporated with a metal or metal-containing solution or solutions. Impregnation is achieved by either the incipient wetness technique or a technique using absorption from a dilute or concentrated solution(s) with subsequent filtration or evaporation to effect total uptake of the metallic components.

In combining the metals with the support, virtually any soluble compound of the respective metals can be used, but a soluble compound which can be easily thermally decomposed is preferred, such as inorganic salts of carbonates, bicarbonates, nitrates, inorganic complex compounds, or organic salts such as a complex salt of acetylacetone, an amine salt, or the like.

To prepare the feed stream, DIB is diluted with any convenient gas such that the molar concentration of DIB in the feed stream that is fed to the reactor is about 75% or less, preferably the molar concentration of DIB is about 50% or less, and most preferably is about 20% or less. Generally, the diluent gas is selected from the group consisting of methane, ethane, and mixtures thereof, but other suitable diluent gases include but are not limited to nitrogen and argon. The diluted DIB feed is contacted in the vapor phase with a dehydrogenation catalyst comprising chromium and/or platinum in a dehydrogenation reactor, resulting in a stream of reactor effluent that contains xylene.

In one embodiment of the process of this invention, the reactor effluent may be sent to a liquid-vapor separation system wherein hydrogen and low molecular weight gases (C₁ to C₂ hydrocarbons) are separated overhead, while the unvaporized products are withdrawn as a liquid. The liquid products may then be sent to a first fractional distillation column where any isobutylene and/or isobutane present are removed overhead, and the unvaporized higher boiling materials may be discharged to a second fractional distillation column. In this second column, any trimethylpentane (TMP) present and any unreacted DIB, which may be one or both of 2,2,4-trimethylpentene-1 (TMPE-1) and 2,2,4-trimethylpentene-2 (TMPE-2), are removed overhead, and the unvaporized higher boiling materials may be sent to a third fractional distillation column. In this third column, toluene and benzene are removed overhead, and the unvaporized aromatics that are recovered contain xylene, of which at least 85% by weight is para-xylene, with the remainder made up of a mixture of the ortho and meta isomers.

Any TMPE-1, TMPE-2 and/or TMP that is recovered overhead from the second column may be recycled to the dehydrogenation reactor. In various embodiments, however, it may be desirable to run the reaction in the substantial absence of, and preferably the absence of, TMP, or to at least provide a feed stream to the dehydrogenation reactor from which TMP is substantially absent, and is preferably absent. For example, if TMP is not completely absent, its presence may be limited to less than 5 mol%, or less than 1 mol%, or less than 0.5 mol%, or to a trace amount as an impurity.

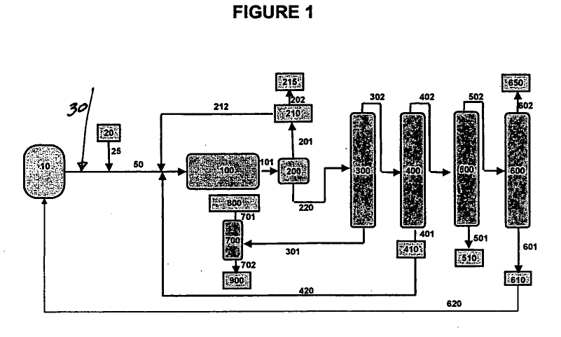

FIG. 1 illustrates various other alternative embodiments of this invention. With reference to this figure, a feed comprising DIB and a diluent gas is fed into a dehydrogenation reactor (100) through a line (50). The feed of DIB through line (50) may come from dimerization zone reactor (10), or an original feed of DIB may instead come from an external source through line (30), or a feed from both sources may be employed. A dehydrogenation catalyst such as chromium and/or platinum resides in reactor (100). The effluent from reactor (100) is sent through line (101) to a flash drum or other liquid-vapor separator system (200). A gas stream comprising hydrogen, and any methane and/or ethane present, is removed overhead through line (201) into a gas separation unit (210) wherein some of any methane and/or ethane recovered is sent to reactor (100) through line (212). Hydrogen exits the gas separation unit (210) through line (202) and is captured in tank (215) for further use. The unvaporized products are sent through to the aromatics column (300) through line (220), whereupon the aromatics are separated and fed through line (301) into a fractionating column (700). Toluene is removed from the fractionating column (700) through line (701) to tank (800). Xylene, preferably p-xylene of at least 85 wt % purity, is removed through line (702) to tank (900) from which further purification can be optionally performed. The toluene in tank (800) can be stored or otherwise used.

The vaporized products from the aromatics column (300), which may include unreacted DIB (such as TMPE-1 and/or TMPE-2) and/or TMP, are removed through line (302) and fed into a fractionating column (400). The unvaporized material, which may include unreacted DIB (such as TMPE-1 and/or TMPE-2) and/or TMP, is removed through line (401) to tank (410), and is then fed from there through line (420) back to the feed stream in line (50). The vaporized material is withdrawn through line (402) and sent to a fractionating column (500) where benzene is separated out through line (501) and sent to tank (510) for storage or further use. The vaporized products are removed from column (500) through line (502) and sent to a fractionating column (600) where any C₃'s present are separated through line (602) to tank (650). The unvaporized material, which is typically primarily C₄'s, is withdrawn from the tank through line (601) and sent to a debutanizer (610) to remove n-butane, after which any isobutane and/or isobutylene remaining is withdrawn through line (620) and forwarded to dimerization zone reactor (10).

The reactor shown as dimerization zone reactor (10) can be any convenient reactor for this purpose, examples of which include reactors such as slurry phase, trickle bed, gas phase, catalytic distillation and the like. The dimerization of isobutylene and/or isobutane to DIB can be effected using a number of catalysts, which are held in the dimerization zone reactor. Examples of effective catalysts include sulfonic acid; cation exchange resins [(e.g., those that contain sulfonic acid groups such as Amberlyst 15; Ostion KS (H+form)]; supported and unsupported metal oxides and mixed metal oxides, including silica-alumina-nickel oxides, titanium dioxide, nickel oxides on alumina, hydrogen-containing boron oxide compounds, bismuth oxides, phosphorous oxides; sodium or lithium metals or compounds supported on a porous potassium salt; t-butanol; zeolites; and sulfuric acid.

In a further alternative embodiment, isobutane may be fed from tank (20) through line (25) into line (50) and from there into dehydrogenation reactor (100). The isobutane is dehydrogented to isobutylene in reactor (100), and will largely pass through the system in that form, will be collected in tank (610), and will be recycled to dimerization reactor (10) for conversion to DIB.

In those embodiments where DIB is fed through line (30) from an external source, DIB may be provided at the external source in a variety of known ways. A preferred means of providing DIB is to dimerize isobutylene. Isobutylene may in turn be provided for such purpose by processes including but not limited to the cracking of methyl tertiary butyl ether (MTBE), the dehydration of isobutanol, butene skeletal isomerization, and the dehydrogenation of isobutane.

The dehydrogenation process in reactor (100) is performed at pressures generally between about 1 and about 5 atmospheres (about 30 to about 150 inches Hg), and is preferably performed at pressures generally above 1 to about 5 atmospheres (above 30 to about 150 inches Hg). The reaction in dimerization zone reactor (10) is performed at temperatures generally between about 5° C. and about 300° C.

The xylene recovered from the reactor effluent is desirably at least 85% by weight para-xylene. The concentration of the para isomer can be increased by various processes such as crystallization, which can be applied to the extent necessary to give xylene that is at least 95 weight percent para-xylene, and preferably at least 98 weight percent para-xylene.

If desired, the process described above may be extended by utilizing the xylene prepared from the dehydrocyclization reaction to make terephthalic acid. This may be done by the oxidation of xylene, preferably para-xylene. In turn, the terephthalic acid may if desired be utilized to make polyester. This may be done by contacting the terephthalic acid as a monomer in a polymerization reaction with another monomer suitable for condensation such as ethylene glycol. The polymerization may be performed by any of a variety of known methods such as the melt polymerization processes such as the transesterification process and the direct esterification process, the solution polymerization process and the solid polymerization process. If desired, the terephthalic acid may be first converted to a terephthalate or a terephthaloyl halide.

As used herein, the term reactor refers to a reaction chamber or tank and the inlet and outlet lines associated therewith. Moreover, it will be recognized that, since the drawings are representative, additional equipment, such as pressure and temperature sensors, pressure relief and control valves, compressors, pumps, storage tanks and the like, may be desired for a commercial plant. The provision of such ancillary items would be in accordance with conventional chemical engineering practice.

Without further elaboration, it is believed that the artisan can, using the description herein, utilize the present invention to its fullest extent. The embodiments in the following examples are, therefore, to be construed as merely illustrative, and do not constrain the remainder of the specification in any way whatsoever.

### EXAMPLES

### General Procedure for Catalyst Testing

Catalyst tests are performed in a fixed-bed continuous-flow quartz reactor with 6.4 mm ID. The catalyst charge varies from 0.5 to 2.0 mL of −10/+60 mesh (−2.00/+0.25 mm) granules. The reactor tube is heated in a tube furnace to 500° C. in a stream of flowing nitrogen until the temperature is stable. A thermocouple inside the catalyst bed is used to measure temperature. Once the desired temperature is achieved, DIB is pumped and vaporized into the flowing diluent stream and passed over the catalyst bed for 5 minutes. Molar concentrations of DIB range from 10 to 75% with the balance being diluent. Contact times vary from 1 to 4 seconds. The entire product stream is analyzed on-line using sampling valves and an HP5890 chromatograph (TCD)/HP 5971 mass selective detector. After 5-60 minutes on stream, the feed is switched to nitrogen only, to quickly purge, and then air is passed over the catalyst at a flow of about 100 cc/minute to burn coke off the catalyst surface. After air treatment, the catalyst is purged with nitrogen. After completion of the nitrogen purge, DIB and diluent gas are introduced back into the stream for the next analysis.

The catalysts as described in Examples 1 to 28 below are tested according to the method described above.

### Example 1

KOH (5.9 g) and CrO₃ (55.5 g) are dissolved in distilled water (100 mL). To this solution is added Davison eta-alumina pellets (10 g) which are then soaked for six hours. After draining, the impregnated pellets are fired to 500° C. for six hours. Chemical analysis by inductively coupled plasma (ICP) of the pellets gives 1.53% K and 13.0% Cr.

### Example 2

A 1 M aqueous solution of Cr(NO₃)₃.9H₂O (53.263 mL) is added simultaneously with 0.1 M HCl (5.326 mL) to a 4.67 M preformed AlO_{1.5} aquasol (11.41 mL) available from the Nyacol Corporation (Nyacol Al-20). The material appears gel-like within minutes. It is dried under vacuum for 5 hours (120° C.) and is dried and then calcined at 300° C. in air for 3 hours prior to use. The material is pelletized and granulated on −10/+20 mesh (−2.0/+0.84 mm) screens prior to use.

### Examples 3 and 4

KOH (3.54 g) and CrO₃ (33.3 g) are dissolved in distilled water (60 mL). To this solution is added 7.5 g of UCI eta-alumina pellets which are then soaked for 21 hours. After draining, the impregnated pellets are fired to 500° C. for six hours in air. Chemical analysis by inductively coupled plasma (ICP) of the pellets gives 1.51% K and 12.0% Cr.

### Examples 5 and 6

KOH (2.36 g) and CrO₃ (22.20 g) are dissolved in distilled water (20 mL). To this solution is added Davison eta-alumina pellets (10 g) which are then soaked for six hours at 75° C. After draining, the impregnated pellets are fired to 500° C. for six hours in air. Chemical analysis by inductively coupled plasma (ICP) of the pellets gives 1.97% K and 18.0% Cr.

### Example 7

NaOH (0.42 g) and CrO₃ (5.55 g) are dissolved in distilled water (10 mL). To this solution is added United Catalysts Inc. (UCI) eta-alumina pellets (10 g) which are then soaked for 21 hours. After draining, the impregnated pellets are fired to 500° C. for six hours in air. Chemical analysis by inductively coupled plasma (ICP) of the pellets gives 1.09% Na and 16.8% Cr.

### Examples 8 and 9

CrO₃ (8.325 g) is dissolved in distilled water (15 mL). To this solution is added UCI eta-alumina pellets (15 g) which are then soaked for 21 hours. After draining, the impregnated pellets are fired to 500° C. for six hours in air. A sample of the above pellets (5 g) is then soaked for three hours in a solution (1.60 g) of 50% by weight CsOH solution diluted to a total volume of 5 mL with distilled water. After draining, the impregnated pellets are fired to 500° C. for three hours in air. Chemical analysis by inductively coupled plasma (ICP) of the pellets gives 7.85% Cs and 5.77% Cr.

### Example 10

CrO₃ (8.325 g) is dissolved in distilled water (15 mL). To this solution is added UCI eta-alumina pellets (15 g) which are then soaked for 21 hours. After draining, the impregnated pellets are fired to 500° C. for six hours in air. A sample (5 g) of the above pellets is then soaked for three hours in a solution (1.09 g) of a 50% by weight RbOH solution diluted to a total volume of 5 mL with distilled water. After draining, the impregnated pellets are fired to 500° C. for three hours in air. Chemical analysis by inductively coupled plasma (ICP) of the pellets gives 4.60% Rb and 10.4% Cr.

### Example 11

CrO₃ (5.55 g) is dissolved in distilled water (10 mL). To this solution is added UCI eta-alumina pellets (10 g) which are then soaked for 21 hours. After draining, the impregnated pellets are fired to 500° C. for three hours in air. The pellets are then soaked for three hours in a solution of KOH (0.59 g) dissolved in distilled water (10 mL). After draining, the impregnated pellets are fired to 500° C. for three hours. Chemical analysis by inductively coupled plasma (ICP) of the pellets gives 2.28% K and 10.8% Cr.

### Example 12

KOH (0.197 g), Fe(NO₃)₃.9H₂O (0.709 g), and CrO₃ (1.932 g) are dissolved in distilled water (2.56 mL). To this solution is added UCI eta-alumina pellets (7.162 g) which are then tumbled on a rotary evaporator for 1 hour. Low heat and vacuum are then applied for sufficient time to completely dry the sample. The pellets are fired to 500° C. for six hours in air. Chemical analysis by inductively coupled plasma (ICP) of the pellets gives 1.45% K, 10.4% Cr, and 1.09% Fe.

### Example 13

KOH (5.9 g) and CrO₃ (55.5 g) are dissolved in distilled water (100 mL). To this solution is added Davison eta-alumina pellets (10 g) which are then soaked for six hours at 75° C. After draining, the impregnated pellets are fired to 500° C. for six hours in air. Chemical analysis by inductively coupled plasma (ICP) of the pellets gives 1.97% K and 18.0% Cr.

### Example 14

A 1 M aqueous solution (36.33 mL) of Cr(NO₃)₃.9H₂O is added simultaneously with 0.1 M HCl (10.81 mL) to 4.67 M preformed AlO_{1.5} aquasol (23.157 mL) available from the Nyacol corporation (Nyacol Al-20). The material becomes gel-like in appearance within minutes. It is dried under vacuum for 5 hours (120° C.) and calcined at 300° C. in air for 3 hours. The material is then pelletized and granulated on −10/+20 mesh (−2.0/+0.84 mm) screens prior to use.

### Example 15

A 1.689 M (with respect to chromium) aqueous solution (81.72 mL) of Cr₃ (OH)₂ (CH₃COO)₇ is added to 118.28 mL of 4.68 M Nyacol Al-20 alumina colloid. A gel point is reached almost immediately. Ethanol (300 mL) is added to this material in order to exchange H₂O (12 hours). The liquid layer is decanted from this mixture after 12 hours. Additional ethanol (400 mL) is added to the gel to allow it to further exchange with water; the material is exchanged overnight, and the top layer is decanted. The ethanol-containing gel is then supercritically dried according to the following procedure in a 1 liter autoclave: Heat for 4 hours to 330° C., 3345 PSIG (23.16 MPa); isotherm 1 hour at 330° C., approximately 3350 PSIG (23.19 MPa); vent while maintaining approximately 330° C. to atmospheric pressure. The free-flowing powder material is pelletized/granulated at 20,000 PSIG (138 MPa) and sieved on −10/+20 mesh (−2.0/+0.84 mm) screens prior to use.

### Examples 16 and 17

A solution of chromium nitrate (19.0 g) dissolved in water (50 mL) is added to eta-alumina (10 g). The pH of the slurry is adjusted (with vigorous stirring) to 9.6 with 1 M sodium hydroxide solution, pausing between additions to assure the pH has stabilized before continuing. The eta-alumina and chromium hydrous oxide precipitate is kept at ambient temperature for 4 hours, then filtered and washed with distilled water (about 200 mL). The suction-dried solid is calcined at 250° C. in flowing air for 2 hours before use.

### Example 18

A 2.56 M (with respect to chromium) aqueous solution (17.373 mL) of Cr₃(OH)₂(CH₃COO)₇ is added to 32.627 mL (2.045 M) of pre-formed ZrO2 colloid (Nyacol, Zr 10/20). The mixture is dried at 120° C. in vacuum for 5 hours prior to use. It is pelletized at 20,000 PSIG (138 MPa) and granulated, −10/+20 mesh (−2.0/+0.84 mm) prior to use.

### Example 19

CrO3 (8.325 g) is dissolved in distilled water (15 mL.). To this solution is added UCI eta-alumina pellets (15 g) which are then soaked for 21 hours. After draining, the impregnated pellets are fired to 500° C. for six hours in air. A sample (5 g) of the above pellets is then soaked for three hours in a solution (0.36 g) of LiNO₃ in distilled water (5 mL). After draining, the impregnated pellets are fired to 500° C. for three hours in air. Chemical analysis by inductively coupled plasma (ICP) of the pellets gives 0.18% Li and 11.5% Cr.

### Example 20

A 0.1 M aqueous solution of SnCl₄ (1.276 mL) is added to 1.5 grams of a preformed and presieved/granulated K/Cr/eta-alumina catalyst prepared as described in Examples 3 and 4. The material is calcined at 375° C. for 3 hours in air prior to use.

Examples 21 and 22

A 0.136 M (with respect to tungsten) aqueous solution of (NH₄)₁₀W₁₂O₄₁.5H₂O (0.6047 mL) is added to 1.5 g of preformed, pre-sieved/granulated K/Cr/eta-alumina catalyst prepared as described in Examples 3 and 4. The material is calcined at 375° C. for 3 hours in air prior to use.

### Example 23

KOH (0.59 g), CrO₃ (5.0 g), and La(NO₃)₃.6H₂O (1.72 g) are dissolved in distilled water (10 mL). To this solution is added UCI eta-alumina pellets (10 g) which are then soaked for 24 hours at room temperature. After draining, the impregnated pellets are fired to 500° C. for three hours in air. Chemical analysis by inductively coupled plasma (ICP) of the pellets gives 1.27% K, 8.72% Cr, and 1.45% La.

### Example 24

KOH (0.59 g), CrO₃ (5.0 g), and Fe(NO₃)₃.9H₂O (1.55 g) are dissolved in distilled water (10 mL). To this solution is added UCI eta-alumina pellets (10 g) which are then soaked for 24 hours at room temperature. After draining, the impregnated pellets are fired to 500° C. for three hours in air. Chemical analysis by inductively coupled plasma (ICP) of the pellets gives 1.45% K, 8.48% Cr and 0.80% Fe.

### Examples 25 And 26

A 0.1 M aqueous SnCl₄ solution (0.126 mL) is added to 1.5 grams of preformed, presieved/granulated K/Cr/eta-alumina catalyst prepared as described in Examples 3 and 4. The material is calcined at 375° C. for 3 hours in air prior to use.

### Example 27

Cr(NO₃)₃.9H₂O (49.80 g) is dissolved in a zirconyl nitrate solution (68.73 g, “20% ZrO2”) and water (18.37 g). Zirconium hydroxide (254.42 g) is mixed with methylcellulose (7.56 g). The solution is mixed with the powder to form a paste. The paste is extruded into ⅛″ (3.2 mm) cylinders. After drying, the extrudates are heated slowly to 500° C. and held at that temperature for 4 hours. The extrudates are then crushed to between 40 and 60 mesh (0.42 mm to 0.25 mm) and slurried in a large excess of a 6% KNO₃ solution for 5 minutes. After filtering and drying, the granules are fired again at 500° C. for 4 hours before use.

### Example 28

A 2.56 M (with respect to chromium) aqueous Cr₃(OH)₂(CH₃CO₂)₇ solution (3.906 mL) is added to 2.039 g of eta-alumina (Engelhard, SNL6469-30-1 ). The material is dried and calcined at 375° C. for 3 hours in air. The material is then granulated and sieved prior to use (−10/+20 mesh (−2.0/+0.84 mm)).

When the catalysts as described in Examples 1 to 28 above are tested according to the method described above, the range of DIB conversion, the range of isobutylene selectivity, the range of xylene selectivity, and the percent para isomer obtained are all at desirable levels.

### Example 29

### Effect of Varying Disobutylene Concentration on Yields to Desired Products

The General Procedure for Catalyst Testing described above is used. The catalyst is 1.53%K/13.0%Cr/eta-alumina prepared as described in Example 1. DIB and diluent flow rates are changed to achieve DIB concentrations, respectively, of 10, 20, 30, 40 and 50 mole percent. The contact time is 3.2 seconds, the reactor temperature is 500° C., and the time on stream before analysis is constant as the DIB concentrations are varied. The range of DIB conversion, the range of isobutylene selectivity, the range of xylene selectivity, and the percent para isomer obtained in this example are all at desirable levels.

1. A non-oxidative process for the manufacture of xylene, comprising:
(a) feeding to a reactor a reactor feed comprising diisobutylene, and a diluent gas selected from the group consisting of methane, ethane and mixtures thereof; and
(b) contacting, in the vapor phase, the reactor feed with a dehydrogenation catalyst in a reactor to produce a stream of reactor effluent that comprises xylene.

2. The process of claim 1 wherein the molar concentration of diisobutylene in the reactor feed is about 75% or less.

3. The process of claim 1 wherein diisobutylene is contacted with the dehydrogenation catalyst at a pressure between about 1 and about 5 atmospheres.

4. The process of claim 1 wherein the dehydrogenation catalyst comprises chromium or platinum.

5. The process of claim 1 wherein the dehydrogenation catalyst comprises chromium.

6. The process of claim 1 wherein the dehydrogenation catalyst is treated with a metal selected from the group consisting of iron, tin, and tungsten.

7. The process of claim 4 wherein the dehydrogenation catalyst further comprises at least one metal selected from the group consisting of sodium, potassium, rubidium, cesium, magnesium, calcium, strontium, and barium.

8. The process of claim 1 wherein the dehydrogenation catalyst is supported on an inorganic oxide selected from the group consisting of alumina, eta-alumina, and zirconia.

9. The process of claim 1 wherein the dehydrogenation catalyst comprises chromium and potassium supported on eta-alumina.

10. The process of claim 1 further comprising a step of recovering xylene from the effluent stream and purifying the xylene by crystallization to increase the content therein of the para isomer.

11. The process of claim 10 wherein the step of crystallization is applied to the extent of providing xylene that contains at least 95 weight percent of the para isomer.

12. The process of claim 1 wherein the effluent stream further comprises unreacted diisobutylene, and the unreacted diisobutylene is recovered from the effluent stream and is recycled to the reactor feed.

13. The process of claim 1 wherein the effluent stream further comprises hydrogen, and the hydrogen is recovered from the effluent stream.

14. The process of claim 1 wherein trimethylpentane is substantially absent from the reactor feed.

15. The process of claim 1 wherein diisobutylene is contacted with the dehydrogenation catalyst in the substantial absence of trimethylpentane.

16. The process of claim 1 further comprising a step of recovering para-xylene from the effluent stream and converting the para-xylene to terephthalic acid.

17. The process of claim 16 further comprising a step of converting the terephthalic acid to polyester.

18. The process of claim 1 wherein the reactor feed further comprises isobutane.

19. The process of claim 18 wherein the effluent stream further comprises isobutylene and unreacted isobutane; and the isobutylene and unreacted isobutane are recovered from the effluent stream and passed to a dimerization zone where they are reacted to produce a mixture comprising diisobutylene.

20. The process of claim 19 wherein the mixture produced in the dimerization zone is recycled to the reactor feed stream.

21. The process of claim 19 wherein the isobutylene and unreacted isobutane are reacted in the dimerization zone at a temperature from about 5° C. to about 300° C.

22. The process of claim 19 wherein the isobutylene and unreacted isobutane are reacted in the dimerization zone in the presence of a catalyst that is selected from the group consisting of sulfuric acid, sulfonic acid, cation exchange resins, supported and unsupported metal oxides or mixed metal oxides, sodium or lithium metals or compounds supported on a porous potassium salt; and zeolites.

23. The process of claim 22 wherein the catalyst is sulfuric acid.

24. The process of claim 22 wherein the cation exchange resin is a sulfonic acid resin.

25. The process of claim 22 wherein the supported and unsupported metal oxides or mixed metal oxides are selected from the group consisting of silica-alumina-nickel oxides, titanium dioxide, nickel oxides on alumina, hydrogen-containing boron oxide compounds, bismuth oxides, and phosphorous oxides.
